(19) United States
(12) Patent Application Publication
(10) Pub. No.: US 2014/0235678 A1
(43) Pub. Date: Aug. 21, 2014

(54) Topical Ophthalmological Pharmaceutical Composition containing Sorafenib

(71) Applicants: Michael Böttger, Wuppertal (DE); Julia Freundlieb, Dortmund (DE); Claudia Hirth-Dietrich, Wuppertal (DE); Jürgen Klar, Wuppertal (DE); Uwe Muenster, Wulfrath (DE); Andreas Ohm, Neuss (DE); Bernd Riedl, Wuppertal (DE)

(72) Inventors: Michael Böttger, Wuppertal (DE); Julia Freundlieb, Dortmund (DE); Claudia Hirth-Dietrich, Wuppertal (DE); Jürgen Klar, Wuppertal (DE); Uwe Muenster, Wulfrath (DE); Andreas Ohm, Neuss (DE); Bernd Riedl, Wuppertal (DE)

(73) Assignee: BAYER HEALTHCARE LLC, Whippany, NJ (US)

(21) Appl. No.: 14/129,557

(22) PCT Filed: Jun. 26, 2012
(86) PCT No.: PCT/EP2012/062354

§ 371 Date: May 2, 2014

(30) Foreign Application Priority Data
Jun. 28, 2011 (EP) 11171715.3
Mar. 29, 2012 (EP) 12161989.4

(51) Int. Cl. A61K 9/00; A61K 31/255; A61K 31/44

(52) U.S. Cl. CPC A61K 9/0048; A61K 31/44; A61K 31/255
USPC 514/350

(57) ABSTRACT

The present invention relates to topical ophthalmological pharmaceutical compositions containing sorafenib or a pharmaceutically acceptable salt thereof or a polymorph, hydrate or solvate thereof and its process of preparation and its use for treating ophthalmological disorders.

The present invention relates to topical ophthalmological pharmaceutical compositions containing sorafenib or a pharmaceutically acceptable salt thereof or a polymorph, hydrate or solvate thereof and its process of preparation and its use for treating ophthalmological disorders.

Sorafenib which is 4{4-[3-(4-chloro-3-trifluoromethylphenyl)-ureido]-phenoxy}-pyridine-2-carboxylic acid methylamide, a compound of formula (I)

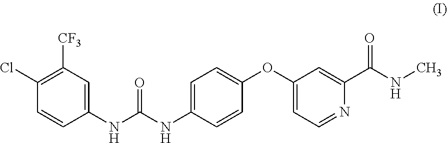 [SMILES: CNC(=O)c1cc(Oc2ccc(NC(=O)Nc3ccc(Cl)c(C(F)(F)F)c3)cc2)ccn1]

is a potent anti-cancer and anti-angiogenic agent (WO 00/042012) that possesses various activities including inhibitory activity on the VEGFR, PDGFR, raf, p38, and/or flt-3 kinase signaling molecules (WO 2004/113274, WO 2005/000284) and it can be used in treating various diseases and conditions like hyper-proliferative disorders such as cancers. Furthermore the tosylate salt of sorafenib and its stable polymorphic form (polymorph I) are disclosed in WO 2006/034797.

Age-related macular degeneration (AMD) is a leading cause of blindness in the elderly population and is recognized as dry and wet AMD (Expert Opin. Ther. Patents (2010), 20(1), 103-11). The dry, or nonexudative, form involves both atrophic and hypertrophic changes of the retinal pigment epithelium (RPE). The dry form is characterized by macular drusen which are pigmented areas containing dead cells and metabolic products that distort the retina and eventually cause loss of acute vision. Patients with nonexudative AMD (dry form) can progress to the wet, or exudative or neovascular, AMD, in which pathologic choroidal neovascular membranes (CNVM) develop under the retina, leak fluid and blood, and, ultimately, cause a centrally blinding disciform scar over a relatively short time frame if left untreated. Choroidal neovascularization (CNV), the growth of new blood vessels from the choroid capillary network across the Bruch's membrane/RPE interface into the neural retina, results in retinal detachment, subretinal and intraretinal edema, and scarring.

Access to the choroid which is between the sclera and the retina other than via the blood is difficult. The eye is composed of three major anatomic compartments, the anterior chamber, posterior chamber, and vitreous cavity, that have limited physiological interaction with each other. The retina is located in the back of the vitreous cavity, and is protected from the outside by the sclera which is the white, tough, impermeable wall of the eye. Choroidal blood flow is the usual method of carrying substances to the choroid and requires e.g. oral or intravenous administration of the drug. Most drugs cannot be delivered to the choroid by eye drops or a depot in vicinity to the eye. Some drugs have been delivered to the retina and thus to the choroid by injection into the vitreous chamber of the eye.

VEGF (vascular endothelial growth factor) is a key cytokine in the development of normal blood vessels as well as the development of vessels in tumors and other tissues undergoing abnormal angiogenesis and appears to play a central role in the pathogenesis of CNV formation (Expert Opin. Ther. Patents (2010), 20(1), 103-118, Expert Opin. Ther. Patents (2009), 18(10), 1573-1580, J. Clin. Invest. (2010), 120(9), 3033-3041, J. Cell. Physiol. (2008), 216, 29-37, New Engl. J. Med. 2006, 355, 1474-1485, WO 2010/127029, WO 2007/064752). Drugs which block the effects of VEGF are described for treating wet AMD such as aptamers like pegaptanib (New Engl. J. Med. 2004, 351, 2805-2816), or VEGF antibodies like ranibizumab (New Engl. J. Med. 2006, 355, 1419-1431) or bevacizumab (Ophthalmology, 2006, 113, 363-372). However, said drugs have to be administered intravitreally by injection into the eye. Sorafenib, a VEGF inhibitior as well, is described for treating CNV by oral administration (Clinical and Experimental Ophthalmology, 2010, 38, 718-726). Pazopanib, a VEGF inhibitior as well, is described for treating AMD by topical administration of eye drops containing an aqueous solution of Pazopanib (WO 2011/009016). WO 2006/133411 describes compounds for the treatment of CNV by topical administration of liposomal formulations. WO 2007/076358, US2006257487 describe aqueous ophthalmological formulations for topical administration. WO 2008/27341 describes emulsions for topical administration to the eye. Young-Hoon P. et al. (Clinical and Experimental Ophthalmology, 2010, 38, 718-726) describes the effect of sorafenib on CNV by oral administration.

Despite the progress described in the art there remains a need for improved medicines for the treatment of ophthalmological disorders like AMD. In particular, there remains a need for topical ophthalmological pharmaceutical compositions like eye drops which can be administered easily and therefore would increase the patient's compliance. The topical ophthalmological pharmaceutical composition has to provide a concentration of the active agent in the eye which is sufficient for an effective therapy. This is dependent on the solubility and the release behavior of the active agent. In the case of a liquid formulation the dissolution properties and chemical stability of the active agent are of importance. In order to support a high compliance the topical ophthalmological pharmaceutical composition should not have to be taken in more than 5 times a day, the less the better. Type and amount of the excipients in combination with the process of preparation of the pharmaceutical composition are essential for release properties, bioavailability of the active agent in the eye, in particular in the back of the eye (e.g. in the area of the retina, Bruch's membrane and choroid), stability and the industrial applicability of the manufacturing process for the topical ophthalmological pharmaceutical composition.

The problem to be solved by the present invention is to provide a topical ophthalmological pharmaceutical composition comprising sorafenib as active agent which has a sufficient stability and which achieves an effective concentration of sorafenib in the eye, in particular in the back of the eye for the treatment of ophthalmological disorders by avoiding an intravenous or oral administration or injection into or close to the eye (e.g. intravitreal or other injections).

Surprisingly the pharmaceutical composition according to the invention provides by topical administration a sufficient amount of the active agent into the eye which is effective for treating ophthalmological disorders. In particular, the pharmaceutical composition according to the invention provides the active agent in a sufficient amount into the back of the eye, i.e. that the pharmaceutical composition according to the invention effects the transportation of the active agent from the front of the eye to the back of the eye. Furthermore the pharmaceutical composition according to the invention has a sufficient stability without any meaningful degradation of the active agent.

The present invention pertains to a topical ophthalmological pharmaceutical composition comprising sorafenib, the compound of the formula (I),

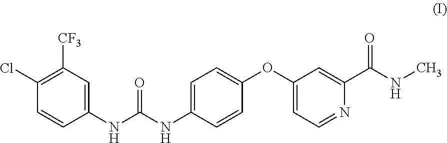 [SMILES: CNC(=O)c1cc(Oc2ccc(NC(=O)Nc3ccc(Cl)c(C(F)(F)F)c3)cc2)ccn1]

or a pharmaceutically acceptable salt of sorafenib, or a polymorph, hydrate or solvate thereof and at least one pharmaceutically acceptable vehicle and optionally at least one pharmaceutically acceptable excipient.

A pharmaceutically acceptable vehicle or excipient is any vehicle or excipient which is relatively non-toxic and innocuous to a patient at concentrations consistent with effective activity of the active agent so that any side effects ascribable to the vehicle or excipient do not vitiate the beneficial effects of the active agent. The term “the compound of formula (I)” or “sorafenib” refer to 4-{4-[({[4-chloro-3-(trifluoromethyl)phenyl]amino}carbonyl)amino]-phenoxy}-N-methylpyridine-2-carboxamide as depicted in formula (I).

The term “compound of the invention” or “active agent” refer to sorafenib or pharmaceutically acceptable salt of sorafenib, or a polymorph, hydrate or solvate thereof.

Solvates for the purposes of the invention are those forms of the compounds or their salts where solvent molecules form a stoichiometric complex in the solid state and include, but are not limited to for example ethanol and methanol.

Hydrates are a specific form of solvates, where the solvent molecule is water. Hydrates of the compounds of the invention or their salts are stoichiometric compositions of the compounds or salts with water, such as, for example, hemi-, mono- or dihydrates. Preference is given to the tosylate of sorafenib.

Salts for the purposes of the present invention are preferably pharmaceutically acceptable salts of the compounds according to the invention. Suitable pharmaceutically acceptable salts are well known to those skilled in the art and include salts of inorganic and organic acids, such as hydrochloric acid, hydrobromic acid, sulfuric acid, phosphoric acid, methanesulphonic acid, trifluoromethanesulfonic acid, benzenesulfonic acid, p-toluenesulfonic acid (tosylate salt), 1-naphthalenesulfonic acid, 2-naphthalenesulfonic acid, acetic acid, trifluoroacetic acid, malic acid, tartaric acid, citric acid, lactic acid, oxalic acid, succinic acid, fumaric acid, maleic acid, benzoic acid, salicylic acid, phenylacetic acid, and mandelic acid. In addition, pharmaceutically acceptable salts include salts of inorganic bases, such as salts containing alkaline cations (e.g., Li⁺ Na⁺ or K⁺), alkaline earth cations (e.g., Mg⁺², Ca⁺² or Ba⁺²), the ammonium cation, as well as acid salts of organic bases, including aliphatic and aromatic substituted ammonium, and quaternary ammonium cations, such as those arising from protonation or peralkylation of triethylamine, N,N-diethylamine, N,N-dicyclohexylamine, lysine, pyridine, N,N-dimethylaminopyridine (DMAP), 1,4-diazabiclo[2.2.2]octane (DABCO), 1,5-diazabicyclo[4.3.0]non-5-ene (DBN) and 1,8-diazabicyclo[5.4.0]undec-7-ene (DBU). Preference is given to the tosylate salt of sorafenib, more preferably the stable polymorphic form (polymorph I) of tosylate salt of sorafenib as disclosed in WO 2006/034797.

Preferred are sorafenib and the tosylate of sorafenib, most preferred is sorafenib tosylate as compounds of the present invention.

The topical ophthalmological pharmaceutical composition according to the invention comprises the compound of the invention, preferably sorafenib, more preferably sorafenib tosylate.

Optionally the topical ophthalmological pharmaceutical composition according to the invention comprises the compound of the invention in a solid form, preferably in a crystalline form, more preferably in a microfine crystalline form.

Micronization can be achieved by standard milling methods, preferably by air jet milling, known to a skilled person. The micronized form can have a mean particle size of from 0.5 to 10 μm, preferably from 1 to 6 μm, more preferably from 2 to 3 μm. The indicated particle size is the mean of the particle size distribution measured by laser diffraction known to a skilled person (measuring device: HELOS, Sympatec).

The minimum concentration of the compound of the invention, preferably sorafenib, more preferably sorafenib tosylate in the topical ophthalmological pharmaceutical composition is 0.1%, preferably 0.2% by weight of the total amount of the composition. The maximum concentration of the compound of the invention, preferably sorafenib, more preferably sorafenib tosylate in the topical ophthalmological pharmaceutical composition is 10%, preferably 5%, more preferably 3% by weight of the total amount of the composition.

Preference is given to a concentration of the compound of the present invention in the pharmaceutical composition from 0.1 to 100 mg/ml, preferably from 1 to 50 mg/ml, more preferably from 2 to 40 mg/ml.

Particular preference is given to a concentration of sorafenib in the pharmaceutical composition from 0.1 to 100 mg/ml, preferably from 1 to 50 mg/ml, more preferably from 2 to 40 mg/ml.

Particular preference is given to a concentration of sorafenib tosylate in the pharmaceutical composition from 0.1 to 100 mg/ml, preferably from 1 to 50 mg/ml, more preferably from 2 to 40 mg/ml.

The topical ophthalmological pharmaceutical composition according to the invention includes but is not limited to eye drops, gels, ointments, dispersions, solutions or suspensions.

One embodiment of the present invention is a topical ophthalmological pharmaceutical composition which is a solution or suspension comprising the compound of the invention, preferably sorafenib, more preferably sorafenib tosylate and an applicable pharmaceutically acceptable vehicle, and optionally one or more pharmaceutically acceptable excipients.

Suitable pharmaceutically acceptable vehicles according to the present invention include but are not limited to oleoyl polyethyleneglycol gylcerides, linoleoyl polyethyleneglycol gylcerides, lauroyl polyethyleneglycol gylcerides, hydrocarbon vehicles like liquid paraffin, light liquid paraffin, soft paraffin (vaseline), hard paraffin, vegetable fatty oils like castor oil, peanut oil or sesame oil, synthetic fatty oils like middle chain trigylcerides, wool alcohols like cetylstearylalcohols, wool fat, glycerol, propylene glycol, polyethyleneglycols (PEG), water like an aqueous isotonic sodium chloride solution or a mixture of thereof, preferably oleoyl polyethyleneglycol glycerides, hydrocarbon vehicles, fatty oils or a mixture of thereof, most preferably hydrocarbon vehicles like liquid paraffin or light liquid paraffin or a mixture thereof.

The pharmaceutically acceptable vehicle is the basis of the topical ophthalmological pharmaceutical composition according to the present invention and is present in the composition in a minimum concentration of 75%, preferably 80%, more preferably 85% and in a maximum concentration of 99.9%, preferably 99%, more preferably 98% by weight of the total amount of the composition.

Suitable further pharmaceutically acceptable excipients used in the topical ophthalmological pharmaceutical composition according to the present invention include but are not limited to surfactants, polymer base carriers like gelling agents, organic co-solvents, pH active components, osmotic active components and preservatives.

Suitable surfactants used in the topical ophthalmological pharmaceutical composition according to the present invention include but are not limited to lipids such as phospholipids, phosphatidylcholines, cardiolipins, fatty acids, phosphatidylethanolamines, phosphatides, tyloxapol, polyethylenglycols and derivatives like PEG 400, PEG 1500, PEG 2000, poloxamer 407, poloxamer 188, polysorbate 80, polysorbate 20, sorbitan laurate, sorbitan stearate, sorbitan palmitate or a mixture thereof, preferably polysorbate 80.

Suitable polymer base carriers like gelling agents used in the topical ophthalmological pharmaceutical composition according to the present invention include but are not limited to cellulose, hydroxypropylmethylcellulose (HPMC), hydroxypropylcellulose (HPC), carboxymethyl cellulose (CMC), methylcellulose (MC), hydroxyethylcellulose (HEC), amylase and derivatives, amylopectins and derivatives, dextran and derivatives, polyvinylpyrrolidone (PVP), polyvinyl alcohol (PVA), and acrylic polymers such as derivatives of polyacrylic or polymethacrylic acid like HEMA, carbopol or a mixture thereof.

Suitable organic co-solvents used in the pharmaceutical composition according to the invention include but are not limited to ethylene glycol, propylene glycol, N-methyl pyrrolidone, 2-pyrrolidone, 3-pyrrolidinol, 1,4-butanediol, dimethylglycol monomethylether, diethyleneglycol monomethylether, solketal, glycerol, polyethylene glycol, polypropylene glycol.

Suitable pH active components such as buffering agents or pH-adjusting agents used in the pharmaceutical composition according to the invention include but are not limited to disodium phosphate, monosodium phosphate, boric acid, sodium borate, sodium citrate, hydrochloric acid, sodium hydroxide.

The pH active components are chosen based on the target pH for the composition which generally ranges from pH 4-9.

Suitable osmotic active components used in the pharmaceutical composition according to the invention include but are not limited to sodium chloride, mannitol, glycerol.

Preservatives used in the pharmaceutical composition according to the invention include but are not limited to benzalkonium chloride, alkyldimethylbenzylammonium chloride, cetrimide, cetylpyridinium chloride, benzododecinium bromide, benzethonium chloride, thiomersal, chlorobutanol, benzyl alcohol, phenoxethanol, phenylethyl alcohol, sorbic acid, methyl and propyl parabens, chlorhexidine digluconate, EDTA or mixtures thereof.

Gelling agents, pH active agents and osmotic active agents are preferably used in the case of an aqueous pharmaceutically acceptable vehicle.

The amount of the suitable further pharmaceutically acceptable excipient in the composition according to the present invention can be from 0.1 to 15%, preferably from 0.5 to 10%, more preferably from 1 to 5% by the total weight of the composition.

Preferably the amount of hydroxypropylmethylcellulose in the composition according to the present invention can be from 0.05 to 15%, preferably from 0.1 to 10%, more preferably from 1 to 5% by the total weight of the composition.

Preferably the amount of polysorbate 80 in the composition according to the present invention can be from 0.05 to 10%, preferably from 0.1 to 7%, more preferably from 0.5 to 4% by the total weight of the composition.

The total amount of the active agent to be administered via the topical route into the eye using the pharmaceutical composition of the present invention will generally range from about 0.01 to 50 mg, preferably 0.02 to 10 mg, more preferably 0.05 to 5 mg per administration and per eye. Based upon standard laboratory techniques known to evaluate compounds useful for the treatment of ophthalmological disorders, by standard pharmacological assays for the determination of treatment of the conditions identified above in mammals, and by comparison of these results with the results of known medicaments that are used to treat these conditions, the effective dosage of the pharmaceutical compositions of this invention can readily be determined by those skilled in the art. The amount of the administered active ingredient can vary widely according to such considerations as the particular compound and dosage unit employed, the mode and time of administration, the period of treatment, the age, sex, and general condition of the patient treated, the nature and extent of the condition treated, the rate of drug metabolism and excretion, the potential drug combinations and drug-drug interactions, and the like.

The pharmaceutical composition according to the invention is administered one or more, preferably up to 5, more preferably up to 3 times per day.

The typical method of administration of the pharmaceutical composition according to the invention is the topical delivery into the eye.

Nevertheless, it may in some cases be advantageous to deviate from the amounts specified, depending on individual response to the active ingredient, type of preparation and time or interval over which the administration is effected. For instance, less than the aforementioned minimum amounts may be sufficient in some cases, while the upper limit specified has to be exceeded in other cases. In the case of administration of relatively large amounts, it may be advisable to divide these into several individual doses over the day.

This pharmaceutical composition will be utilized to achieve the desired pharmacological effect by preferably topical administration into the eye to a patient in need thereof, and will have advantageous properties in terms of drug release, bioavailability, and/or compliance in mammals. A patient, for the purpose of this invention, is a mammal, including a human, in need of treatment for the particular condition or disease.

The pharmaceutical composition according to the invention is chemically stable for more than 18 months, preferably more than 24 months. Chemically stable according the present invention means that the active agent does not degrade significantly during storage.

Process for Manufacturing

Various methods can be used to prepare the ophthalmological pharmaceutical composition according to the invention. First the pharmaceutically acceptable vehicle is prepared by optionally mixing the applicable vehicle or mixture of vehicles with the pharmaceutically acceptable excipients.

Thereafter the active agent is dispersed, solved or suspended into said mixture. The process may also include sterilization e.g. by sterile precipitation, gamma irradiation, sterile filtration, heat sterilization, aseptic filling, or a combination of such optional steps.

The present invention also relates to a process for the manufacturing of a topical ophthalmological pharmaceutical composition according to the invention, wherein the compound of the present invention is dispersed, solved or suspended in an applicable pharmaceutically acceptable vehicle optionally in the presence of further one or more pharmaceutically acceptable excipients and the mixture is homogenized.

Preference is given to a process for the manufacturing of a topical ophthalmological pharmaceutical composition according to the invention, wherein
a) the applicable pharmaceutically acceptable vehicle or a mixture of applicable pharmaceutically acceptable vehicles is prepared by mixing the vehicles optionally in the presence of a further one or more pharmaceutically acceptable excipients,
b) the compound of the present invention, preferably sorafenib, more preferably sorafenib tosylate, is dispersed, solved or suspended into said applicable pharmaceutically acceptable vehicle for example at room temperature, optionally in the presence of a further one or more pharmaceutically acceptable excipients,
c) the mixture is homogenized by stiffing, shaking or vortexing, preferably stirring, at room temperature,
d) the mixture is subdivided into single units and filled into applicable vials, container, tube, flask, dropper and/or syringe.

Optionally in step a) the further one or more pharmaceutically acceptable excipients are added to the applicable pharmaceutically acceptable vehicle at elevated temperatures for example of 40 to 70° C.

Method of Treating Ophthalmological Disorders

The present invention also relates to a use of the pharmaceutical composition according to the invention to treat or prevent ophthalmological disorders.

Examples of ophthalmological disorders according to the invention include but are not limited to age-related macular degeneration (AMD), choroidal neovascularization (CNV), retinal detachment, diabetic retinopathy, atrophic changes of the retinal pigment epithelium (RPE), hypertrophic changes of the retinal pigment epithelium (RPE), diabetic macular edema, retinal vein occlusion, choroidal retinal vein occlusion, macular edema, macular edema due to retinal vein occlusion, angiogenesis in the front of the eye like corneal angiogenesis following e.g. keratitis, corneal transplantation or keratoplasty, corneal angiogenesis due to hypoxia (extensive contact lens wearing), pterygium conjunctivae, subretinal edema and intraretinal edema.

Examples of age-related macular degeneration (AMD) include but are not limited to dry or nonexudative AMD, or wet or exudative or neovascular AMD.

Preference is given to age-related macular degeneration (AMD) like dry AMD, wet AMD or choroidal neovascularization (CNV).

The pharmaceutical composition according to the invention can be administered as the sole pharmaceutical composition or in combination with one or more other pharmaceutical compositions or active agents where the combination causes no unacceptable adverse effects.

“Combination” means for the purposes of the invention not only a dosage form which contains all the active agents (so-called fixed combinations), and combination packs containing the active agents separate from one another, but also active agents which are administered simultaneously or sequentially, as long as they are employed for the prophylaxis or treatment of the same disease.

Since the combination according to the invention is well tolerated and is potentially effective even in low dosages, a wide range of formulation variants is possible. Thus, one possibility is to formulate the individual active ingredients of the combination according to the invention separately. In this case, it is not absolutely necessary for the individual active ingredients to be taken at the same time; on the contrary, sequential intake may be advantageous to achieve optimal effects. It is appropriate with such separate administration to combine the formulations of the individual active ingredients simultaneously together in a suitable primary packaging. The active ingredients are present in the primary packaging in each case in separate containers which may be, for example, tubes, bottles or blister packs. Such separate packaging of the components in the joint primary packaging is also referred to as a kit.

In one embodiment, the pharmaceutical compositions of the present invention can be combined with other ophthalmological agents. Examples of such agents include but are not limited to cartenoids like lycopene, lutein, zeaxanthin, phytoene, phytofluene, carnosic acid and derivatives thereof like carnosol, 6,7-dehydrocarnosic acid, 7-ketocarnosic acid, a zink source like zinc oxide or a zinc salt like its chloride, acetate, gluconate, carbonate, sulphate, borate, nitrate or silicate salt, copper oxide, vitamin A, vitamin C, vitamin E and/or B-carotene.

In another embodiment, the pharmaceutical compositions of the present invention can be combined with other signal transduction inhibitors targeting receptor kinases of the domain families of e.g. VEGFR, PDGFR, FGFR and their respective ligands or other pathway inhibitors like VEGF-Trap (aflibercept), pegaptanib, ranibizumab, pazopanib, bevasiranib, KH-902, mecamylamine, PF-04523655, E-10030, ACU-4429, volociximab, sirolismus, fenretinide, disulfiram, sonepcizumab and/or tandospirone. These agents include, by no way of limitation, antibodies such as Avastin (bevacizumab). These agents also include, by no way of limitation, small-molecule inhibitors such as STI-571/Gleevec (Zvelebil, Curr. Opin. Oncol., Endocr. Metab. Invest. Drugs 2000, 2(1), 74-82), PTK-787 (Wood et al., Cancer Res. 2000, 60(8), 2178-2189), SU-11248 (Demetri et al., Proceedings of the American Society for Clinical Oncology 2004, 23, abstract 3001), ZD-6474 (Hennequin et al., 92nd AACR Meeting, New Orleans, March 24-28, 2001, abstract 3152), AG-13736 (Herbst et al., Clin. Cancer Res. 2003, 9, 16 (suppl 1), abstract C253), KRN-951 (Taguchi et al., 95th AACR Meeting, Orlando, Fla., 2004, abstract 2575), CP-547,632 (Beebe et al., Cancer Res. 2003, 63, 7301-7309), CP-673,451 (Roberts et al., Proceedings of the American Association of Cancer Research 2004, 45, abstract 3989), CHIR-258 (Lee et al., Proceedings of the American Association of Cancer Research 2004, 45, abstract 2130), MLN-518 (Shen et al., Blood 2003, 102, 11, abstract 476), and AZD-2171 (Hennequin et al., Proceedings of the American Association of Cancer Research 2004, 45, abstract 4539), PKC412, nepafenac.

Preference is given to a combination with bevacizumab, aflibercept, pegaptanib, ranibizumab, pazopanib and/or bevasiranib.

Generally, the use of the other ophthalmological agents in combination with the pharmaceutical compositions of the present invention will serve to:

(1) yield better efficacy as compared to administration of either agent alone,

(2) provide for the administration of lesser amounts of the administered agents,

(3) provide for treating a broader spectrum of mammals, especially humans,

(4) provide for a higher response rate among treated patients,

(5) yield efficacy and tolerability results at least as good as those of the agents used alone, compared to known instances where other agent combinations produce antagonistic effects. It is believed that one skilled in the art, using the preceding information and information available in the art, can utilize the present invention to its fullest extent.

It should be apparent to one of ordinary skill in the art that changes and modifications can be made to this invention without departing from the spirit or scope of the invention as it is set forth herein.

All publications, applications and patents cited above and below are incorporated herein by reference.

The weight data are, unless stated otherwise, percentages by weight and parts are parts by weight.

### EXAMPLES

### Example 1

### Ophthalmological Suspension Comprising Sorafenib Tosylate in Oleoyl Polyethyleneglycol Glyceride (20 mg/ml)

200 mg of micronized sorafenib tosylate is mixed to oleoyl polyethyleneglycol glyceride (10 ml). The mixture is homogenized by stiffing at room temperature for 15 minutes.

### Example 2

### Ophthalmological Suspension Comprising Sorafenib Tosylate in Liquid Paraffin (20 mg/ml)

400 mg of micronized sorafenib tosylate is mixed in 20 ml of light liquid paraffin. The mixture is homogenized by stirring at room temperature for 15 minutes.

### Example 3

### Ophthalmological Composition Comprising Sorafenib Tosylate in Water Based Vehicle (20 mg/ml)

1.7 g of hydroxypropymethylcellulose 15 cp (HPMC) is dispersed in isotonic sodium chloride solution (48 g, 0.9% NaCl in water) at 70° C. The mixture is cooled down to room temperature while stiffing. At room temperature evaporated water, and subsequently polysorbate 80 (0.5 g) is added and dissolved under moderate stiffing. 518 mg of sorafenib tosylate is added to an aliquot of the prepared vehicle (24.5 g) and the suspension is homogenized by gently stiffing at room temperature for 15 minutes.

### Example 4

### Topical Efficacy of a Composition containing Sorafenib in the Laser-Induced Choroidal Neovascularization (CNV) Model

The aim of this study is to determine whether twice daily topical administration (eye drops) of the topical ophthalmological pharmaceutical compositions according to the invention results in a decrease of vascular leakage and/or choroidal neovascularization in a rat model of laser-induced choroidal neovascularisation (Dobi et al, Arch. Ophthalmol. 1989, 107(2), 264-269 or Frank et al, Curr. Eye Res. 1989 March, 8(3), 239-247)

For this purpose, pigmented Brown-Norway rats with no visible sign of ocular defects are selected. On day 0, the animals are anaesthetized by an intraperitoneal injection (15 mg/kg xylazine and 80 mg/kg ketamine (dissolved in water containing 5 mg/ml chlorobutanol hemihydrate and propylenglycol) After instillation of one drop of 0.5% atropin (dissolved in 0.9% saline containing Benzalkoniumchloride) to dilate the pupils, choroidal neovascularisation is induced by burning six holes in the retina (disruption of Bruch's membrane) of one eye per animal (lesion size: 50 μm, laser intensity: 150 mW; stimulus duration: 100 ms) using a 532 nm argon laser. Of each test formulation (formulation containing the example according to the invention and vehicle formulation without active agent), 10 μl are applied to the affected eye twice daily at an 10:14 hour interval during the complete observation period of 23 days. The body weight of all animals is recorded before the start and once daily during the study. An angiography is performed on day 21 using a fluorescence fundus camera (Kowe Genesis Df, Japan). Here, after anesthesia and pupillary dilation, 10% sodium fluorescein (dye, dissolved in water) is subcutaneously injected and pictures are recorded 2 and 10 min after dye injection. The vascular leakage of the fluorescein on the angiograms is evaluated by three different examiners who are blinded for group allocation (example versus respective vehicle). Each lesion is scored with 0 (no leakage) to 3 (strongly stained), and a mean from all 6 lesions is used as the value for the respective animal. On day 23, animals are sacrificed and eyes are harvested and fixed in 4% paraformaldehyde solution for 1 hour at room temperature. After washing, the retina is carefully peeled, and the sclera-choroid complex is washed, blocked and stained with a FITC-isolectine B4 antibody in order to visualize the vasculature. Then, the sclera-choroids are flat-mounted and examined under a fluorescence microscope (Keyence Biozero) at 488 nm excitation wavelength. The area (in μm²) of choroidal neovascularization is measured using ImageTool software.

### RESULTS

Labrafil (n=8 per group)

| | Vascular leakage | Choroidal neo- |
|---|---|---|
| | [angiography | vascularization |
| | score] | lesion size [μm²] |
| 100% oleoyl polyethyleneglycol | 1.81 ± 0.25 | 92388 ± 20123 |
| glycerides (vehicle control) | | |
| Sorafenib (20 mg/ml) suspension | 1.13 ± 0.21 | 65207 ± 11972 |
| in 100% oleoyl polyethyleneglycol | | |
| glycerides (example 1) | | |
| p-value | <0.001 | 0.006 |

Paraffin (n=8 per group)

| | Vascular leakage | Choroidal neo- |
|---|---|---|
| | [angiography | vascularization |
| | score] | lesion size [μm²] |
| 100% paraffin | 1.92 ± 0.27 | 89976 ± 18448 |
| Sorafenib (20 mg/ml) suspension | 1.47 ± 0.19 | 58254 ± 14769 |
| in 100% paraffin (example 2) | | |
| p-value | 0.002 | 0.002 |

Water-based vehicle (8 per group)

| | Vascular leakage | Choroidal neo- |
|---|---|---|
| | [angiography | vascularization |
| | score] | lesion size [μm²] |
| Water-based vehicle | 2.03 ± 0.16 | 91839 ± 8906 |
| Sorafenib (20 mg/ml) suspension | 1.77 ± 0.27 | 64010 ± 17747 |
| in water-based vehicle (example 3) | | |
| p-value | 0.037 | 0.001 |

Although the invention has been disclosed with reference to specific embodiments, it is apparent that other embodiments and variations of the invention may be devised by others skilled in the art without departing from the true spirit and scope of the invention. The claims are intended to be construed to include all such embodiments and equivalent variations.

1. A topical ophthalmological pharmaceutical composition comprising sorafenib or a pharmaceutically acceptable salt of sorafenib, or a polymorph, hydrate or solvate thereof as active agent and at least one pharmaceutically acceptable vehicle.

2. The pharmaceutical composition of claim 1 containing sorafenib tosylate as active agent.

3. The pharmaceutical composition of claim 1 wherein the concentration of the active agent in the pharmaceutical composition is from 0.1 to 10% by weight of the total amount of the composition.

4. The pharmaceutical composition of claim 1 wherein the concentration of the active agent in the pharmaceutical composition is from 0.1 to 100 mg/ml.

5. The pharmaceutical composition of claim 1 in form of eye drops, gels, ointments, dispersions, solutions or suspensions.

6. The pharmaceutical composition of claim 5 comprising an applicable pharmaceutically acceptable vehicle.

7. The pharmaceutical composition of claim 6 wherein the pharmaceutically acceptable vehicle is selected from the group comprising oleoyl polyethyleneglycol gylcerides, linoleoyl polyethyleneglycol gylcerides, lauroyl polyethyleneglycol gylcerides, liquid paraffin, light liquid paraffin, soft paraffin (vaseline), hard paraffin, castor oil, peanut oil, sesame oil, middle chain trigylcerides, cetylstearylalcohols, wool fat, glycerol, propylene glycol, polyethyleneglycols (PEG) or a mixture of those, water or a mixture thereof.

8. A combination comprising the pharmaceutical composition according to claim 1 combined with one or more further active agents.

9. A process for manufacturing a pharmaceutical composition according to claim 1 wherein the active agent is mixed in an applicable pharmaceutically acceptable vehicle optionally in the presence of further one or more pharmaceutically acceptable excipients and the mixture is homogenized.

10. The pharmaceutical composition of claim 1 for the use of treating or preventing an ophthalmological disorder selected from the group comprising age-related macular degeneration (AMD), choroidal neovascularization (CNV), retinal detachment, diabetic retinopathy, atrophic changes of the retinal pigment epithelium (RPE), hypertrophic changes of the retinal pigment epithelium (RPE), diabetic macular edema, retinal vein occlusion, choroidal retinal vein occlusion, macular edema, macular edema due to retinal vein occlusion, angiogenesis in the front of the eye, corneal angiogenesis following keratitis, corneal transplantation or keratoplasty, corneal angiogenesis due to hypoxia (extensive contact lens wearing), pterygium conjunctivae, subretinal edema and intraretinal edema.

11. The pharmaceutical composition of claim 10 for the use of treating or preventing an ophthalmological disorder selected from the group comprising dry AMD, wet AMD or choroidal neovascularization (CNV).

12. Method for using the pharmaceutical composition according to Claim 1 to treat or prevent an ophthalmological disorder selected from the group comprising age-related macular degeneration (AMD), choroidal neovascularization (CNV), retinal detachment, diabetic retinopathy, atrophic changes of the retinal pigment epithelium (RPE), hypertrophic changes of the retinal pigment epithelium (RPE), diabetic macular edema, retinal vein occlusion, choroidal retinal vein occlusion, macular edema, macular edema due to retinal vein occlusion, angiogenesis in the front of the eye, corneal angiogenesis following keratitis, corneal transplantation or keratoplasty, corneal angiogenesis due to hypoxia (extensive contact lens wearing), pterygium conjunctivae, subretinal edema and intraretinal edema.
